(19) United States
(12) Patent Application Publication
(10) Pub. No.: US 2022/0079480 A1
(43) Pub. Date: Mar. 17, 2022

(54) CONTINUOUS EX-VIVO AFFINITY-BASED SENSING OF INTERSTITIAL FLUID

(71) Applicant: University Of Cincinnati, Cincinnati, OH (US)

(72) Inventors: Jason Charles Heikenfeld, Cincinnati, OH (US); Mark Friedel, Cincinnati, OH (US)

(21) Appl. No.: 17/421,616

(22) PCT Filed: Nov. 13, 2019
(86) PCT No.: PCT/US19/61083

§ 371 Date: Jul. 8, 2021

Related U.S. Application Data
(60) Provisional application No. 62/835,572, filed on Apr. 18, 2019.
(60) Provisional application No. 62/791,393, filed on Jan. 11, 2019.

(51) Int. Cl. A61B 5/1473; A61B 5/145; A61B 5/00

(52) U.S. Cl. CPC A61B 5/14735; A61B 5/685; A61B 5/14514; A61B 5/14546

(57) ABSTRACT

Described are sensing devices and methods that continuously sense at least one analyte in an invasive biofluid are described. The devices include at least one affinity-based sensor with a plurality of probes. The probes include a binding that is specific to the at least one analyte. The device further includes at least one diffusion pathway between the affinity-based sensor and the source of the invasive biofluid.

### CROSS-REFERENCE TO RELATED APPLICATIONS

This application claims priority to, and the benefit of the filing date of, U.S. Provisional Application No. 62/791,393 filed Jan. 11, 2019, the disclosure of which is incorporated by reference herein in its entirety. In addition, this application claims priority to, and the benefit of the filing date of, U.S. Provisional Application No. 62/835,572 filed Apr. 18, 2019, the disclosure of which is incorporated by reference herein in its entirety.

### BACKGROUND OF THE INVENTION

Interstitial fluid contains many of the same analytes as blood and often at comparable concentrations. As a result, interstitial fluid presents an alternative biofluid to blood for detection of analytes such as glucose for diabetes monitoring. Commonly employed practices for continuous monitoring of glucose in interstitial fluid include in-dwelling sensors, where a needle is utilized to insert the sensor into the dermis of the skin, and micro-needles where the sensor is placed ex-vivo and the analyte is coupled from interstitial fluid to the sensor by diffusion to the sensor. In both products and in research, the biosensing of analytes in interstitial fluid monitoring has been dominated by metabolite analytes because electrochemical enzymatic sensors are readily available and well developed for these compounds, and because metabolites are found at generally high concentrations (mM) which simplifies their detection. It is far more difficult to detect analytes with concentrations in the μM, nM, and pM concentration ranges. For these types of analytes, typical enzymatic sensors do not exist.

Affinity-based sensors such as electrochemical or optical aptamers are known to be inherently reversible (truly continuous), and known to provide ranges of detections in the μM or lower ranges in biofluids such as whole blood. These sensors, however, are quite different than enzymatic sensors, which metabolize and therefore consume the analyte. This is because affinity sensors require equilibration of analyte concentration with the sensor itself, and have a known binding affinity for the target analyte. Affinity sensors have been developed for implantable biosensing in fluids such as interstitial fluid and blood, but have not been demonstrated for ex-vivo sensing of invasive biofluids such as blood or interstitial fluid where the analyte reaches the affinity biosensor by diffusion through a fluidic pathway in a device. This may be in part because a major distinction and challenge in lag-time exists for affinity sensors for ex-vivo diffusion-based detection of analytes in invasive biofluids, a challenge that has not been resolved.

To better understand this challenge, consider a hollow or hydrogel microneedle array coupled to the dermis with an ex-vivo enzymatic sensor, such as that developed by Arkal Medical (DOI: 10.1177/1932296814526191). Firstly, the analyte concentration at the sensor can be assumed to be zero or close to zero, because the biosensor consumes the analyte due to the presence of enzymes which rapidly metabolize the analyte. One important feature is the diffusive flux of analytes from the body to the sensor. The sensor signal is proportional to this diffusive flux. Therefore, if the concentration of the analyte in the body increases or decreases, the diffusive flux readily responds due to the laws of diffusion, and the diffusive flux experienced at the sensor responds quickly. Furthermore, because the concentration of the analyte at the sensor is effectively zero, the concentration difference between the analyte in the body and the analyte at the sensor is large, ensuring a strong diffusive flux of the analyte based on the laws of diffusion. None of the above assumptions are true for an affinity-based sensor such as an aptamer sensor.

Consider again a similar device with a conventional hollow or hydrogel microneedle array with an ex-vivo sensor, but the sensor is an affinity-based biosensor. Firstly, for the affinity-based sensor to accurately read concentrations of the analyte in the invasive biofluid, the concentration must equilibrate between the biofluid and the biosensor. In this scenario, a much greater lag time can exist because the affinity sensor must wait for this concentration equilibrium to occur, and unlike an enzymatic sensor, the affinity-based sensor does not benefit from only a change in diffusive flux between the biofluid and the sensor. Furthermore, the difference in concentration between the biofluid and the affinity sensor will often be small compared to the concentration different between the biofluid and an enzymatic sensors, which also limits the diffusive flux according to the laws of diffusion. As a result, the integration of an affinity sensor with a device that performs ex-vivo sensing of an analyte in an invasive biofluid, presents a non-obvious challenge.

To resolve lag times, one might consider coating the ends of microneedles with an affinity-based biosensor, however, this can bring additional challenges beyond issues with lag times. For example, consider a conventional microneedle length of 300 μm which is a length that has been used to minimize perceived pain by companies such as Arkal Medical, which utilized an array of 200 hollow microneedles as reported in Journal of Diabetes Science and Technology, 2014, Vol. 8(3) 483-487, DOI:10.1177/1932296814526191. Increasing the number of microneedles or length of microneedles causes significant increase of perceived pain as reported in Clin J Pain 2008; 24:585-594, DOI: 10.1097/AJP.0b013e31816778f9. Next, consider that the epidermis is ˜100 μm thick on locations such as the forearm. Then consider the effects of skin defects on skin roughness (10's to 100's of μm) and of hair (˜20-200 μm thick). Lastly, consider that skin roughness (peak to valley) heights are ˜100 μm in young adults, and ˜200 μm or more in older adults (Skin Pharmacol Physiol 2016; 29:291-299, DOI: 10.1159/000450760). It is easily feasible that at least one microneedle will not reach the dermis and therefore not be in fluidic communication with interstitial fluid. Furthermore, motion of the body or organs or changing pressures against a device can make the problem of microneedles not being in fluidic communication with interstitial fluid even worse, and can induce motion artifacts. Returning to the consideration of microneedles that are coated with affinity based biosensors, any microneedle not implanted properly into the dermis could give a zero or false signal. Therefore, a significant challenge exists where the affinity-based sensor must be somehow kept in constant fluidic communication with the interstitial fluid in the dermis. Furthermore, simply increasing needle length may not be relevant for many applications (pain, or chance it could insert into subcutaneous fat). Consider medication monitoring, where medication compliance is an issue. Here you may have older adults, with rougher skin, and any microneedle pain may lower compliance which defeats the purpose of monitoring medication concentrations. Furthermore, any prior art techniques utilized for enzymatic sensors is not necessarily relevant to an affinity-based biosensor, because the physics of operation for enzymatic sensors is quite different than that of affinity based biosensors.

### SUMMARY OF THE INVENTION

Many of the drawbacks and limitations stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sensing technology into proximity with biofluid and analytes.

Embodiments of the disclosed invention are directed to continuous ex-vivo affinity-based sensing of analytes in interstitial fluid. Embodiments of the disclosed invention provide sensing systems that resolve lag-time challenges when the analyte is coupled to the sensor by primarily diffusion, and solve issues where the affinity-based biosensor might lose fluidic contact with the dermis.

In an embodiment, a continuous sensing device for at least one analyte in an invasive biofluid is described. The continuous sensing device includes at least one affinity-based sensor with a plurality of probes with binding that is specific to the at least one analyte. The continuous sensing device further includes at least one diffusion pathway between the affinity-based sensor and the source of the invasive biofluid.

Alternatively or in addition, in an embodiment, the affinity-based sensor included in the continuous sensing device is ex-vivo.

Alternatively or in addition, in an embodiment, the majority of the change in analyte concentration that is sensed by the affinity-based sensor is transported to and from the sensor by diffusion, and if the analyte concentration in the biofluid decreases the diffusion of analyte is in the direction back towards the source of analyte.

Alternatively or in addition, in an embodiment, the affinity-based sensor included in the continuous sensing device is an aptamer sensor.

Alternatively or in addition, in an embodiment, the affinity-based sensor included in the continuous sensing device is an electrochemical aptamer sensor.

Alternatively or in addition, in an embodiment, the affinity-based sensor included in the continuous sensing device is an optical aptamer sensor.

Alternatively or in addition, in an embodiment, the continuous the diffusion pathway includes at least one microneedle that provides a pathway for diffusion of the at least one analyte through the dermis.

Alternatively or in addition, in an embodiment, the microneedle is hollow.

Alternatively or in addition, in an embodiment, the sensor is outside of the body and outside the stratum-corneum of the skin.

Alternatively or in addition, in an embodiment, the continuous sensing device includes at least one sample volume adjacent to the sensor, wherein the sample volume is less than one of 10 μL/cm², 5 μL/cm², 2 μL/cm², 1 μL/cm², 0.5 μL/cm², or 0.2 μL/cm².

Alternatively or in addition, in an embodiment, the continuous sensing device has a diffusion lag time for an analyte having a molecular weight less than 1000 Da in molecular weight and a diffusion coefficient at greater than 6E-6 cm2/s, wherein the diffusion lag time is less than at least one of 50 min, 25 min, 10 min, 5 min, 2.5 min, or 1 min

Alternatively or in addition, in an embodiment, the continuous sensing device has a diffusion lag time for an analyte with a diffusion coefficient greater than 1.2E-6 cm²/s, wherein the diffusion lag time is less than at least one of 250 min, 125 min, 50 min, 25 min, 12.5 min, or 5 min.

Alternatively or in addition, in an embodiment, the continuous sensing device has a diffusion lag time for an analyte with a diffusion coefficient greater than 6E-7 cm²/s, wherein the diffusion lag time is less than at least one of 500 min, 250 min, 100 min, 50 min, 25 min, or 10 min.

Alternatively or in addition, in an embodiment, wherein the affinity-based sensor is in fluidic communication with a plurality of microneedles, and in further fluidic communication with the dermis, even if at least one, but not all, microneedle is not in fluidic communication with the dermis.

Alternatively or in addition, in an embodiment, the number of microneedles included in the continuous sensing device is at least one of >10, >20, >50, >100, >200, or >1000 microneedles.

Alternatively or in addition, in an embodiment, wherein said affinity-based sensor probes have an attached redox couple which generates the signal change.

Alternatively or in addition, in an embodiment, the affinity-based sensor is in-dwelling.

In another embodiment, a continuous sensing device for at least one analyte in an invasive biofluid is described. The continuous sensing device includes at least one affinity-based sensor with a plurality of probes with binding that is specific to the at least one analyte. The continuous sensing device includes the affinity-based sensor in fluidic communication with a plurality of microneedles, and in further fluidic communication with the dermis, even if at least one, but not all, microneedles are not in fluidic communication with the dermis. The continuous sensing device further includes at least one diffusion pathway between the affinity-based sensor and the source of the invasive biofluid.

Alternatively or in addition, in an embodiment, the number of microneedles is at least one of >10, >20, >50, >100, >200 microneedles.

### BRIEF DESCRIPTION OF THE DRAWINGS

The objects and advantages of the disclosed invention will be further appreciated in light of the following detailed descriptions and drawings in which:

FIG. 1A is a cross-sectional view of a device according to an embodiment of the disclosed invention.

FIG. 1B is a cross-sectional view of a device according to an alternative embodiment of the disclosed invention.

FIG. 2 is a simulation plot of analyte concentration vs. time for the devices of FIG. 1.

FIG. 3 is a simulation plot of analyte concentration vs. time for the devices of FIG. 1.

FIG. 4 is a cross-sectional view of a device according to an alternative embodiment of the disclosed invention.

FIG. 5 is a cross-sectional view of a device according to an alternative embodiment of the disclosed invention.

### DEFINITIONS

As used herein, “invasive biofluid” means one in which the biofluid is accessible through forming a pore into the body (such as a laser-cut hole through the skin), by placing a foreign object into the body (such as a needle or microneedle or other material), or other suitable means and biofluids that are invasive in the manner in which the biofluid is accessed.

As used herein, “ex-vivo” means outside the body or not placed directly within the body. For example, a sensor placed above the epidermis of the skin is ex-vivo. For example, with a needle placed into the body connected to a device or material that is outside the body, in which the sensor is housed inside the needle, the sensor is also ex-vivo because the sensor is mainly facing a foreign object (i.e. the needle) instead of the body (e.g. the dermis) and the sensor is therefore coupled to the biofluid only through a foreign (man-made) fluidic pathway. A sensor that is coated with a hydrogel or other membrane, and that sensor and coating facing directly the inside of the body (e.g. the dermis) would not be ex-vivo. This would be an implanted or in-dwelling sensor, where lag time due to diffusion to the sensor would not benefit from the present invention.

As used herein, “sample” means an invasive biofluid source of analytes. Fluid samples can include blood, interstitial fluid, or other invasive biofluid samples.

As used herein, “sample volume” means the effective total volume between an ex-vivo sensor and an invasive biofluid which effects the lag-time between concentration of an analyte in the biofluid and the concentration at the sensor. This sample volume could be a fluidic or microfluidic volume defined by walls such as channel walls or be defined by a fluidic pathwidth such as that through a hydrogel.

As used herein, “continuous sensing” with a “continuous sensor” means a sensor that reversibly changes in response to concentration of an analyte, where the only requirement to increase or decrease the signal of the sensor is to change the concentration of the analyte in the biofluid. Such a sensor, therefore, does not require regeneration of the sensor by locally changing pH, for example. Similarly, as used herein, “continuous monitoring” means the capability of a device to provide at least one measurement of an analyte in an invasive biofluid determined by a continuous or multiple collection and sensing of that measurement or to provide a plurality of measurements of the analyte over time.

As used herein, “probe” means a molecule or other material that specifically binds to at least one analyte such that upon binding to the analyte the probe induces a local change in the probe such as a change in electrical, chemical, optical, mechanical, or thermal behavior.

As used herein, “affinity-based sensor” means as biosensor that is a continuous sensor with a plurality of probes that reversibly bind to an analyte, which do not consume, metabolize, or otherwise chemically alter the analyte, wherein the binding of analyte to the sensor increases with increasing concentration of the analyte, and the binding of the analyte decreases with decreasing concentration of the analyte.

As used herein, “microfluidic components” are channels in polymer, textiles, paper, hydrogels, or other components known in the art of microfluidics for guiding movement of a fluid or at least partial containment of a fluid.

As used herein, “diffusion” is the net movement of a substance from a region of high concentration to a region of low concentration. This is also referred to as the movement of a substance down a concentration gradient.

As used herein, “diffusion pathway” is a pathway that provides diffusion coupling between an invasive biofluid and a sensor. Said differently, as concentration changes in the biofluid, the sensor receives changes in concentration of the analyte through the diffusive pathway. A diffusion pathway as described herein pertains only to an ex-vivo sensor.

As used herein, “diffusion lag time” is the time required for a change in analyte concentration in an invasive biofluid to reach a sensor by diffusion through a diffusion pathway such that the fluid immediately adjacent to the sensor is at least 90% of the concentration of the concentration in the invasive biofluid.

As used herein, “advective transport” is a transport mechanism of a substance or conserved property by a fluid due to the fluid's bulk motion.

As used herein, “convection” is the concerted, collective movement of groups or aggregates of molecules within fluids and rheids, either through advection or through diffusion or a combination of both.

### DETAILED DESCRIPTION OF THE INVENTION

Embodiments of the disclosed invention are directed to continuous ex-vivo affinity-based sensing of analytes in interstitial fluid. Embodiments of the disclosed invention provide sensing systems that resolve lag-time challenges when the analyte is coupled to the sensor by primarily diffusion.

Certain embodiments of the disclosed invention show sensors as simple individual elements. It is understood that many sensors require two or more electrodes, reference electrodes, or additional supporting technology or features which are not captured in the description herein. Sensors measure a characteristic of an analyte. Sensors are preferably electrical in nature, but may also include optical, chemical, mechanical, or other known biosensing mechanisms. Sensors can be in duplicate, triplicate, or more, to provide improved data and readings. Sensors may provide continuous or discrete data and/or readings. Certain embodiments of the disclosed invention show sub-components of what would be sensing devices with more sub-components needed for use of the device in various applications, which are known (e.g., a battery, antenna, adhesive), and for purposes of brevity and focus on inventive aspects, such components may not be explicitly shown in the diagrams or described in the embodiments of the disclosed invention.

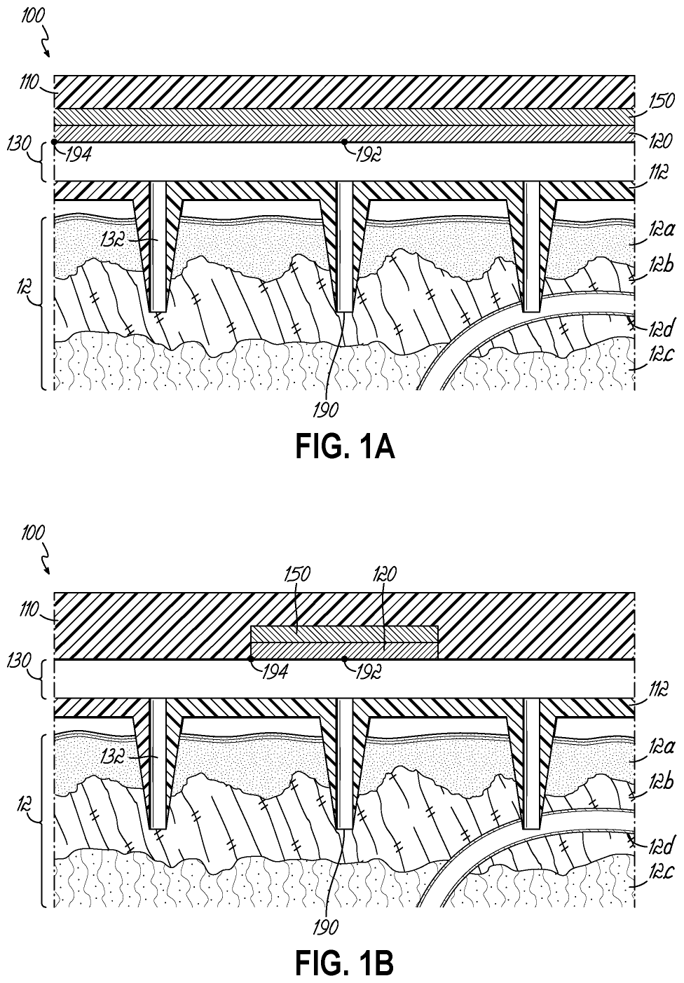

With reference to FIG. 1A, in an embodiment of the disclosed invention, an ex-vivo device 100 is placed partially in-vivo into the skin 12 comprised of the epidermis 12a, dermis 12b, and the subcutaneous or hypodermis 12c. A portion of the device 100 accesses invasive fluids such as interstitial fluid from the dermis 12b and/or blood from a capillary 12d. Access is provided, for example, by microneedles 112 formed of metal, polymer, semiconductor, glass or other suitable material, and may include a hollow lumen 132 that contributes to a sample volume. Sample volume is also contributed to by volume 130 above material from which the microneedles 112 project yet below sensor probes 120 on electrode 150 on a polymer substrate 110. Together, probes 120 and electrode 150 form a sensor 120, 150. Together the volume of volume 130 and lumen 132 form a sample volume and can be a microfluidic component such as channels, a hydrogel, or other suitable material. A diffusion pathway exists from the invasive biofluid such as interstitial fluid or blood to the sensor probes 120, the pathway beginning at location 190 at the inlet to the microneedle 112, first reaching the sensor probes 120 at location 192, and having an ending pathway at location 194. Location 194 is noted as the end because it is simply the contact point on the probe 120 that is the furthest distance from location 190, and can be referred to as the furthest location. Alternative arrangements and materials are possible, such as using a single needle, hydrogel polymer microneedles, or other suitable means to couple an invasive fluid to one or more ex-vivo sensors, although these alternative arrangements and materials are not be explicitly shown in the figures. Sensor probes 120 are affinity-based and could be for example aptamer sequences that are selective in reversible binding to an analyte and permanently thiol bonded to the electrode 150 and used to sense an analyte such as glucose, cortisol, vasopressin, IL-6, a drug, or other analyte by means of electrochemical detection. In some embodiments, the electrode 150 includes gold. Probes could be electrical in nature and utilize an attached redox couple to transduce the electrochemical signal or instead measure change in impedance between the electrode and solution. Probes could also be optical in nature, such as fluorescently labeled aptamers that are labeled with a quencher (i.e. molecular beacon) that may not require electrode 150 but may use optical sensors and light sources to detect analyte aptamer interactions. Such alternative arrangements are not explicitly shown in the figures.

With further reference to FIG. 1A, to illustrate a case that is not an embodiment of the present invention, if the sensor probes 120 were not affinity-based but were instead enzymatic in nature, such as those used for glucose, ethanol, or lactate sensing, an analyte would only need to reach location 192 before it could be properly detected because enzymatic sensors consume, metabolize, or alter the analyte and then measure byproducts or co-factors associated with the enzymatic reaction. Therefore, the analyte concentration at the sensor probes 120 could also be assumed to be zero or close to zero, because the probes consume the analyte due the enzymes which rapidly metabolize the analyte. Therefore, in devices using enzymatic sensors, the concentration of analyte at all of the probes 120 is not important. Rather, the diffusive flux of analytes from the body at location 190 to the sensor 120,150 at location 192 is measured. The sensor 120,150 signal is proportional to this diffusive flux. Therefore, if the concentration of the analyte in the body increases or decreases, the diffusive flux readily responds due to the laws of diffusion, and the diffusive flux experienced at the sensor 120,150 responds quickly. Furthermore, because the concentration of the analyte at the sensor 120,150 is effectively zero, the concentration difference between the analyte in the body at location 190 and the analyte at the sensor location 192 is large, ensuring a strong diffusive flux of the analyte based on the laws of diffusion. None of the above assumptions are true for embodiments of the present invention which involve an affinity-based sensor such as an aptamer sensor.

With further reference to FIG. 1A, to illustrate a case that is an embodiment of the present invention, assume that the sensor 120,150 is an affinity-based biosensor. Firstly, for the affinity-based sensor 120,150 to accurately read concentrations of the analyte in the invasive biofluid, the concentration of the analyte must equilibrate between the biofluid and the sensor 120,150. In this scenario, a much greater diffusion lag time can exist (as compared to enzymatic sensors) because the affinity-based sensor must wait for this concentration equilibrium to occur, and unlike an enzymatic sensor, the affinity-based sensor does not benefit from only a change in diffusive flux between the biofluid and the sensor 120,150. Even more challenging, it will take the longest for changes in concentrations of an analyte at location 190 to reach the furthest location 194, which is important because for the sensor 120,150 to measure an analyte concentration as accurately as possible, the concentration should be uniform at all areas across the surface of the sensor 120,150. If the concentration is not uniform, portions of the sensor 120,150 can give false high or false low readings of analyte concentrations. Furthermore, the difference in analyte concentration between the biofluid and the affinity sensor 120,150 will often be small compared to the concentration difference between the biofluid and an enzymatic sensor, which also limits the diffusive flux according to the laws of diffusion. As a result, the integration of an affinity sensor 120,150 with a device that performs ex-vivo sensing of an invasive biofluid, presents a non-obvious challenge.

With reference to FIG. 1B, FIG. 1B shows an alternative arrangement that is equivalent to the case of FIG. 1A, in order to illustrate that the present invention is not limited to the specific embodiments taught herein.

With further reference to FIG. 1A and/or FIG. 1B, to illustrate challenges in diffusive lag times for an affinity-based sensor 120,150, consider the following examples taught for Cortisol and Glucose, which has the following molecular weight and diffusion coefficients: Cortisol: 362 Da, 2.8E-6 cm2/s; Glucose 180 Da, 6.6E-6 cm2/s. Other analytes are possible, and are not limited to: Vasopressin 1060 Da, 4E-6 cm2/s; Amyloid Beta 3500 Da, 5E-7 cm2/s; RNA 20,000 Da, 1E-6 cm2/s, IL-6: 26,000 Da, 2.7E-7 cm2/s. Even larger analytes include for example antibodies. Cortisol and Glucose will be specifically taught, and diffusion coefficients simply mathematically scaled by 10X to represent other potential analytes.

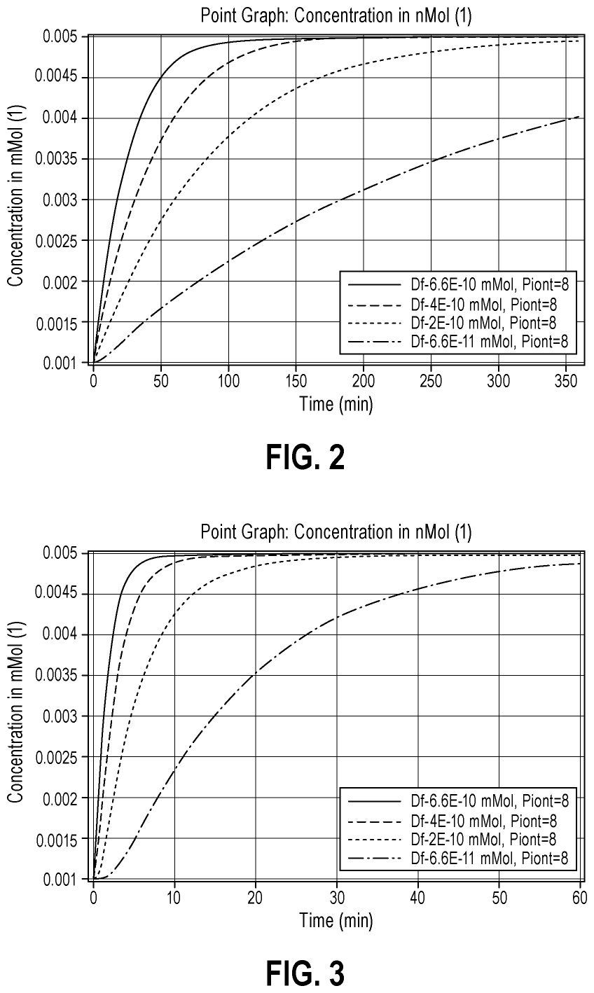

Diffusion coefficient is inversely proportional to the effective ‘radius’ of the solute. At least because mass increases volumetrically (r³), a large change in mass (r³) for an analyte does not result in much change in diffusion coefficient (1/r). To accurately model diffusion using COMSOL as a modeling tool, the equation used for plotting concentration vs. time will be: c(x,t)=c0 Erfc (x/(2 (Dt)\hat{ }½)), where D is the diffusivity and c0 the initial concentration and Erfc is the complementary error function. Two cases are modeled, both for a typical set of hollow microneedle dimensions: 300 μm long and 2500 μm2 cross-sectional lumen (hollow) area in the micro needle tube (e.g. 50×50 μm). This will represent a first area and volume represented in FIG. 1 as hollow lumen 132. Volume 130 adjacent to the sensor 120,150 will be modeled in two cases. The first case, which is plotted in FIG. 2, is for a conventional volume 130 that is 100 μm thick and 150 μm from location 192 to location 194, which is representative of a typical microneedle array and how conventionally a sensor 120,150 would be integrated within the device 100. The second case is for a reduced volume, and is plotted in FIG. 3, and represents reducing the volume 130 by 10×. For simplicity in the modeling, the volume is assumed to be 40 μm thick and 40 μm from location 192 to location 194. This second case is roughly equivalent to a volume that is 10 μm thick and 150 μm wide (10× thinner than the first case). These equivalent cases are permitted with the example used here because it is generally the entire volume 130 that places significant constraint on diffusive lag time. The model data is for glucose (D=6.6E-10 m²/s) and additional curves are shown for lower diffusivities (e.g. scaling to proteins). In the model setup, the biofluid in the dermis starts with a 1 nM concentration at t=0 and at t=0+ abruptly switches to a 5 nM concentration. The modeling results will be the same for 1 pM to 5 pM, etc. because for smaller concentrations diffusion flux is lower, but the final change in concentration is also less. Therefore the results for 1-5 nM are representative of any other concentration change as well (e.g. 7.3 pM to 8.5 pM, will have the same result in diffusive lag time). If the any fluids included in volume 130 or hollow lumen 132 are not ideal fluids, and filled, for example, with a hydrogel, the diffusive lag times for larger analytes will be even slower due to collisions between the analytes and the hydrogel matrix.

With further reference to embodiments of the present invention, thickness of volume 130 can be <100, <50, <20, <10, <5 μm, <2 μm, <1 μm for volumes 130 that are <10 μL/cm2, <5 μL/cm2, <2 μL/cm2, <1 μL/cm2, 0.5 μL/cm2, <0.2 μL/cm2. With further reference to embodiments of the present invention, the present invention also enables diffusion lag times to 90% of concentration in biofluid for an analyte that has a 10X lower diffusion coefficient than glucose of 6.6E-6 cm2/s which is >6E-7 cm2/s (e.g. vasopressin, IL-6, etc.) that is at least one of <500 min, <250 min, <100 min, <50 min, <25 min, <10 min. The present invention also enables diffusion lag times to 90% of concentration in biofluid for an analyte that is <1000 Da (e.g. glucose, cortisol, etc.) with >6E-6 cm2/s that is at least one of <50 min, <25 min, <10 min, <5 min, <2.5 min, <1 min.

As an experimental example, a 3×1 hollow microneedle array over a 2.5×0.6 mm area with a liquid volume capacity of 7.2 nL (representing hollow lumen 132), was combined with a casing of 71.4 nL (representing volume 130), to create an example device with a total filling volume of 78.6 nL. In this case 5 μM cortisol was diffused. Cortisol has a molecular weight of <1000 Da. The time to diffuse 90% (4.5 μM) of cortisol to the sensor was less than 45 minutes. This can be extrapolated to examples where volume increase is directly proportional to lag time increase; therefore, volumes of 10 nL, 100 nL, 500 nL, and, 1 μL would approximately give lag times less than 6, 60, 300, and 600 minutes respectively for analytes <1000 Daltons and a diffusion coefficient >6.6E-6 cm²/s. Furthermore, by increasing microneedle density we can lower diffusive lag time. For example, consider a volume of luL created by a 1 cm² patch with 10 μM thickness. By increasing needle density from 3 microneedles/cm² to 30, 60, 120, 300, 600, or 1500 microneedles/cm² it is possible to achieve diffusive lag times less than 60, 30, 15, 5, 2.5 and 1 minutes or 600, 300, 150, 50, 25, or 10 minutes for analytes with a diffusion coefficient which is >6E-7 cm²/s.

With further reference to embodiments of the present invention, as stated in the background section, having affinity-based sensors coated on the ends of microneedles could cause false signal readings because the sensors could lose contact with dermal interstitial fluid. Therefore, the present invention also enables at least one affinity-based biosensor 120,150 that is in fluidic communication with a plurality of microneedles 112 and which is always kept in fluidic communication with the dermis 12b even if one or more microneedles 112, but not all, lose fluidic contact with the dermis 12b. The embodiments taught in FIG. 1A and FIG. 1B provide this in the case that elements included in volume 130 and hollow lumen 132 stay wet with fluid through capillary action or by being filled with a wicking material such as a hydrogel that stays wet with fluid. Alternatively, the sensor 120 could be coated on microneedle 112, including inside hollow lumen 132, provided that the same condition of being kept in fluidic communication is achieved. To ensure fluidic contact with the dermis 12b, a plurality of microneedles 112 are needed, preferably at least one of >3, >5, >10, >20, >50, >100, >200 microneedles 112.

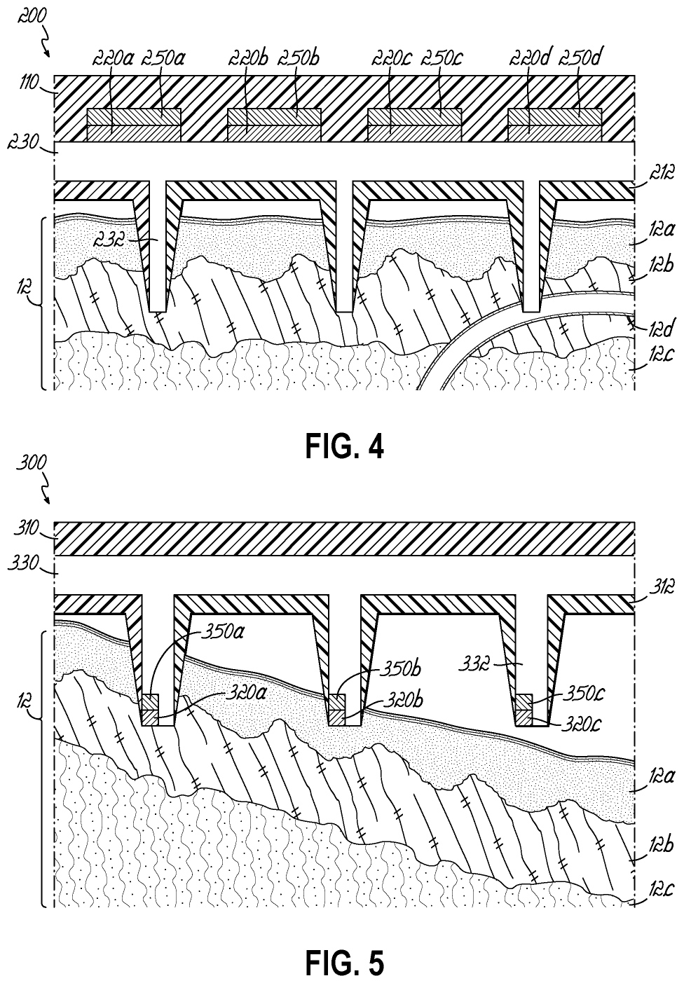

With reference to FIG. 4, where like numerals refer to like features previously shown and described for FIGS. 1A, 1B, and 2, an alternative embodiment of the invention is shown for a device 200. The device 200 is placed partially in-vivo into the skin 12 comprised of the epidermis 12a, dermis 12b, and the subcutaneous or hypodermis 12c. A portion of the device 200 accesses invasive fluids such as interstitial fluid from the dermis 12b and/or blood from a capillary 12d. Access is provided, for example, by microneedles 212 formed of metal, polymer, semiconductor, glass or other suitable material, and may include a hydrogel 232 that contributes to a sample volume. Sample volume is also contributed to by hydrogel 230, which may be a continuation of hydrogel 232, above material from which the microneedles 212 project yet below sensor probes 220a,b,c,d on electrode 250a,b,c,d on a polymer substrate 110. Together, probes 220a,b,c,d and electrodes 250a,b,c,d form sensors 220a,b,c,d, 250a,b,c,d. Together the volume of volume 230 and hollow lumen 232 form a sample volume and can be a microfluidic component such as channels, a hydrogel, or other suitable material. Alternative arrangements and materials are possible, such as using a single needle, hydrogel polymer microneedles, or other suitable means to couple an invasive fluid to one or more ex-vivo sensors, although these alternative arrangements and materials are not be explicitly shown in the figures. Sensor probes 220a,b,c,d are affinity-based and could be for example aptamer sequences that are selective in reversible binding to an analyte and permanently thiol bonded to the electrodes 250a,b,c,d and used to sense an analyte such as glucose, cortisol, vasopressin, IL-6, a drug, or other analyte by means of electrochemical detection. In some embodiments, the electrodes 250a,b,c,d include gold. Probes 220a,b,c,d could be electrical in nature and utilize an attached redox couple to transduce the electrochemical signal or instead measure change in impedance between the electrode and solution. Probes 220a,b,c,d could also be optical in nature, such as fluorescently labeled aptamers that are labeled with a quencher (i.e. molecular beacon) that may not require electrodes 250a,b,c,d but may use optical sensors and light sources to detect analyte aptamer interactions. Such alternative arrangements are not explicitly shown in the figures.

A plurality of sensors or a plurality of surfaces for a single affinity-based biosensor are show as 220a,b,c,d and 250a,b,c,d. All of the plurality of sensor 220a,b,c,d, 250a,b,c,d surfaces are kept in fluid communication with each other, else the signal measured from the sensors 220a,b,c,d, 250a,b,c,d could be incorrect. For example, some sensors 220a,b,c,d, 250a,b,c,d require a 2 or 3 electrode system, and some sensors 220a,b,c,d, 250a,b,c,d might be in duplicate, triplicate, etc. Any sensor 220a,b,c,d, 250a,b,c,d not wetted by fluid, but is nevertheless in communication with fluid in the skin 12 could give a false signal, such as a false low signal. Furthermore, wetting of the sensor 220a,b,c,d, 250a,b,c,d changes with body motion, which can cause body-motion artifacts as well. Therefore the plurality of sensor 220a,b,c,d and 250a,b,c,d surfaces are all in contact with a wicking material or channel such as a hydrogel 230, 232 that is always wet with fluid and/or interstitial fluid.

With reference to FIG. 5, where like numerals refer to like features previously shown and described for FIGS. 1A, 1B, and 2, an alternative embodiment device 300 employs in-dwelling sensors 320a,b,c, 350a,b,c that are in or on microneedles 312, which will have the same requirement to be wetted as described for FIG. 2, and which show a solution to this potential problem of remaining wetted in the form of a hydrogel such as agar 330, 332. Continuing to refer to FIG. 5, an ex-vivo device 300 is placed partially in-vivo into the skin 12 comprised of the epidermis 12a, dermis 12b, and the subcutaneous or hypodermis 12c. A portion of the device 300 accesses invasive fluids such as interstitial fluid from the dermis 12b and/or blood from a capillary 12d. Access is provided, for example, by microneedles 312 formed of metal, polymer, semiconductor, glass or other suitable material, and may include a hydrogel 332 that contributes to a sample volume. Sample volume is also contributed to by hydrogel 330, which may be a continuation of hydrogel 332, above material from which the microneedles 312 project yet below sensor probes 320a,b,c on electrode 350a,b,c on a polymer substrate 310. FIG. 5 also illustrates imperfect contact with the skin where sensor 320a, 350a surfaces, are in proper contact with the dermis 12b, but due to skin roughness or skin defects or incomplete microneedle penetration (as non-limiting examples) sensor 320b, 350b surfaces and sensor 320c, 350c surfaces are not in proper contact with the dermis 12b directly, but are provided indirect contact through hydrogel 330, 332.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

1. A continuous sensing device for at least one analyte in an invasive biofluid, comprising;
at least one affinity-based sensor with a plurality of probes with binding that is specific to the at least one analyte;
and wherein there is at least one diffusion pathway between the affinity-based sensor and the source of the invasive biofluid.

2. The device of claim 1, wherein the affinity-based sensor is ex-vivo.

3. The device of claim 1, where the majority of the change in analyte concentration that is sensed by the affinity-based sensor is transported to and from the affinity-based sensor by diffusion, and if the analyte concentration in the biofluid decreases the diffusion of analyte is in the direction back towards the source of analyte.

4. The device of claim 1 where the affinity-based sensor is an aptamer sensor.

5. The device of claim 4, wherein the affinity-based sensor is an electrochemical aptamer sensor.

6. The device of claim 4, wherein the affinity-based sensor is an optical aptamer sensor.

7. The device of claim 1, wherein the diffusion pathway includes at least one microneedle that provides a pathway for diffusion of the at least one analyte through the dermis.

8. The device of claim 1, wherein the microneedle is hollow.

9. The device of claim 1, wherein the affinity-based sensor is outside of the body and outside the stratum-corneum of the skin.

10. The device of claim 1, including at least one sample volume adjacent to the affinity-based sensor, wherein the sample volume is less than one of 10 μL/cm2, 5 μL/cm2, 2 μL/cm2, 1 μL/cm2, 0.5 μL/cm2, or 0.2 μL/cm2.

11. The device of claim 1, having a diffusion lag time for an analyte with a diffusion coefficient greater than 1.2E-6 cm²/s, wherein the diffusion lag time is less than at least one of 250 min, 125 min, 50 min, 25 min, 12.5 min, or 5 min.

12. The device of claim 1, having a diffusion lag time for an analyte with a diffusion coefficient greater than 6E-7 cm²/s, wherein the diffusion lag time is less than at least one of 500 min, 250 min, 100 min, 50 min, 25 min, or 10 min.

12. The device of claim 1, having a diffusion lag time for an analyte having a molecular weight less than 1000 Da in molecular weight wherein the diffusion lag time is less than at least one of 150 min, 60, 30, 15, 10, 5, 2.5, or 1 min.

14. The device of claim 1, wherein the affinity-based sensor is in fluidic communication with a plurality of microneedles, and in further fluidic communication with the dermis, even if at least one, but not all, microneedle is not in fluidic communication with the dermis.

15. The device of claim 1, wherein the number of microneedles is at least one of >3, >10, >20, >50, >100, >200, >1000 microneedles.

16. The device of claim 1, wherein said affinity-based sensor probes have an attached redox couple which generates the signal change.

17. The device of claim 3 wherein the affinity-based sensor is in-dwelling.

18. A continuous sensing device for at least one analyte in an invasive biofluid, comprising;
at least one affinity-based sensor with a plurality of probes with binding that is specific to the at least one analyte;
wherein the affinity-based sensor is in fluidic communication with a plurality of microneedles, and in further fluidic communication with a dermis, even if at least one, but not all, microneedle is not in fluidic communication with the dermis,
and wherein there is at least one diffusion pathway between the affinity-based sensor and the source of the invasive biofluid.

19. The device of claim 18, wherein the number of microneedles is at least one of >3, >10, >20, >50, >100, >200 microneedles.
